(19) United States
(12) Patent Application Publication
(10) Pub. No.: US 2019/0088374 A1
(43) Pub. Date: Mar. 21, 2019

(54) REMOTE DENTAL CONSULTATION METHOD AND SYSTEM

(71) Applicants: Randal Stuart Elloway, Red Bluff, CA (US); Noura Manouk Elloway, Red Bluff, CA (US)

(72) Inventors: Randal Stuart Elloway, Red Bluff, CA (US); Noura Manouk Elloway, Red Bluff, CA (US)

(21) Appl. No.: 16/134,660

(22) Filed: Sep. 18, 2018

Related U.S. Application Data
(60) Provisional application No. 62/561,073, filed on Sep. 20, 2017.

(51) Int. Cl. G16H 80/00; G16H 30/20

(52) U.S. Cl. CPC G16H 80/00; G16H 30/20

(57) ABSTRACT

A remote dental consultation method and system compatible with a plurality of handheld intra-oral imaging devices is disclosed. The system has the ability to capture imagery from a plurality of intra-oral imaging devices. The system allows dental patients to consult with dentist professionals in real time and receive treatment recommendations based upon images transmitted from devices that patients can remotely operate themselves.

### CROSS-REFERENCE TO RELATED APPLICATION

This application claims the benefit of co-pending U.S. Provisional Patent Application Ser. No. 62/561,073, filed Sep. 20, 2017, which is incorporated herein in its entirety.

### FIELD OF THE INVENTION

The present invention relates generally to the field of dentistry, and more particularly, to a dental consultation method and system.

### BACKGROUND OF THE INVENTION

It is common for individuals to require dental consultation. For example, an individual may have tooth pain or a broken tooth that needs to be evaluated by a dental professional. To receive dental consultation or evaluation, individuals usually need to go to a dental office in-person.

However, in-person dental exams may be expensive and time-consuming for both dentists and patients. Further, it is hard for dental professionals to remotely monitor and follow up with patients, and such in-person visits may increase the likelihood of insurance fraud.

Various online systems have been created to help provide remote consultation to potential dental patients. For example, computers have been involved in dentistry since the late 1970s with the advent of decision support system software (also known as DDS). DDS was developed in an effort to provide the dental practitioner with patient record keeping and treatment plan assistance. Teledentistry at that time used videoconference technologies for remote consultations and when the internet expanded rapidly in the 1990s dentists began developing web-based management platforms to streamline the patient practitioner exchange process. As large, dental imaging devices (such as stationary X-ray machines and cameras) became much smaller and were converted to intra-oral imaging devices in the early 2000s, dental researchers began looking at ways to connect such devices to online networks to streamline interactions between patients, dentists and specialists.

However, currently a system does not exist that provides accurate, convenient, and effective remote consultation to potential patients that also benefits dental professionals and insurers.

Accordingly, there exists a need for an accurate, convenient, and effective remote dental consultation system.

### SUMMARY OF THE INVENTION

This summary is provided to introduce a selection of concepts in a simplified form that are further described below in the detailed description. This summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

Disclosed is a remote dental consultation system, the system comprising, a personal electronic device, and an intra-oral imaging device operatively connected to the personal electronic device, wherein the personal electronic device is configured to communicate images taken by the intra-oral imaging device to a dental professional.

In another aspect, the intra-oral imaging device uses trans-illumination light.

In another aspect, the intra-oral imaging device is an intra-oral camera.

In another aspect, the intra-oral imaging device is an s-ray sonogram device.

In another aspect, the intra-oral imaging device uses x-ray radiation.

Further disclosed is a remote dental consultation system, the system comprising, a personal electronic device, and an intra-oral imaging device operatively connected to the personal electronic device, wherein the personal electronic device is configured to communicate data collected by the intra-oral imaging device to a remote computing system.

In another aspect, the remote computing system is associated with a dental professional.

In another aspect, the intra-oral imaging device is configured to capture external tooth and gum features.

In another aspect, the intra-oral imaging device is configured to capture internal tooth features.

In another aspect, the intra-oral imaging device includes a digital intra-oral camera.

In another aspect, the intra-oral imaging device includes a handheld X-Ray probe.

In another aspect, the intra-oral imaging device further includes an X-Ray pad, and wherein the X-Ray probe is configured to transmit X-Rays through a tooth and onto the X-Ray pad to thereby generate an exposure image that is transmitted to the personal electronic device.

In another aspect, the intra-oral imaging device includes a sonogram device having one or more ultrasound sensors.

In another aspect, the intra-oral imaging device includes a trans-illuminator having a high-intensity LED.

In another aspect, the personal electronic device is further configured to receive, from the remote computing system, remote data indicating a diagnosis made by a dental professional based on the data collected by the intra-oral imaging device.

In another aspect, the data includes video captured by the intra-oral imaging device that is communicated to the remote computing system in real-time.

In another aspect, the imaging device is at least partially integrated in the personal electronic device.

Further disclosed is a personal electronic device, comprising, a logic machine, and a storage machine holding instructions executable by the logic machine to, communicatively couple with an intra-oral imaging device, receive image data from the intra-oral imaging device, the image data capturing one or more dental features of a user, and transmit the image data to a remote computing system associated with a dental professional.

In another aspect, the one or more dental features include external tooth and gum features.

In another aspect, the one or more dental features include internal tooth features.

In another aspect, the intra-oral imaging device includes a digital intra-oral camera.

In another aspect, the intra-oral imaging device includes a handheld X-Ray probe.

In another aspect, the intra-oral imaging device includes a sonogram device having one or more ultrasound sensors.

In another aspect, the intra-oral imaging device includes a trans-illuminator having a high-intensity LED.

Disclosed is a remote dental consultation system, the system comprising, a personal electronic device, and an intra-oral imaging device operatively connected to the personal electronic device, wherein the personal electronic device is configured to communicate image data capturing one or more dental features of a user collected by the intra-oral imaging device to a remote computing system associated with a dental professional, and to receive remote data from the remote computing system indicating a diagnosis made by the dental professional based on the data collected by the intra-oral imaging device.

These and other objects, features, and advantages of the present invention will become more readily apparent from the attached drawings and the detailed description of the preferred embodiments, which follow.

### BRIEF DESCRIPTION OF THE DRAWINGS

The preferred embodiments of the claimed subject matter will hereinafter be described in conjunction with the appended drawings provided to illustrate and not to limit the scope of the claimed subject matter, where like designations denote like elements, and in which:

FIG. 1 presents a front view of a mobile device being connected to various diagnostic devices, where a dental consultation is displayed on a mobile device, in accordance with aspects of the present disclosure;

FIG. 2 schematically presents the system being used with an x-ray imaging peripheral, in accordance with aspects of the present disclosure;

FIG. 3 schematically presents dental consultation methods according to user types, in accordance with aspects of the present disclosure, and

FIG. 4 schematically presents a computing system, in accordance with aspects of the present disclosure.

It is to be understood that like reference numerals refer to like parts throughout the several views of the drawings.

### DETAILED DESCRIPTION

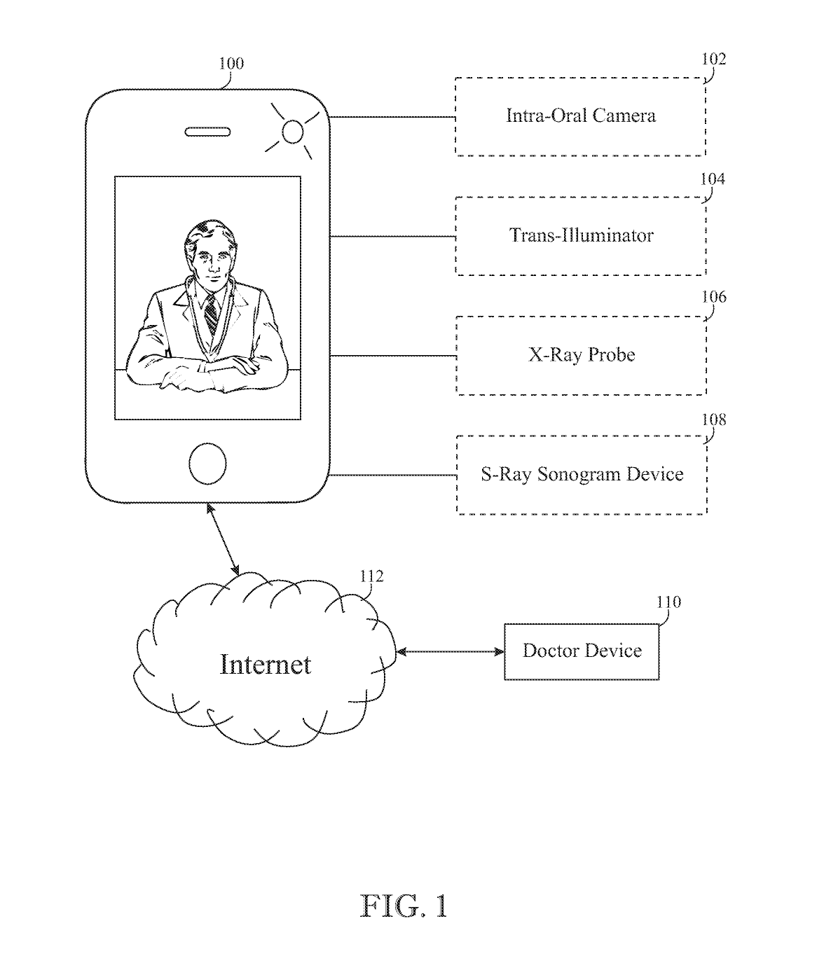

The following detailed description is merely exemplary in nature and is not intended to limit the described embodiments of the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in FIG. 1.

The illustration of FIG. 1 presents a dental consultation system including a mobile communications device 100. The device 100 may run a software application to execute any appropriate method or task described herein. The software application may be written in a code language such as, but not limited to, Java™, C programming and the like and compatible with Linux™, Windows™, Android™ and Apple™ operating system platforms etc. The application may have the ability to run on a multitude of computing hardware devices (including but not limited to laptops, desktops, tablets, personal data assistants, smart phones, cell phones and the like). As described below, the device 100 may include a suitable storage machine holding instructions executable by a suitable logic machine to effect the approaches described herein.

The illustration of FIG. 1 shows a patient's mobile device 100 being operatively connected to one or more handheld, intra-oral imaging devices, including but not limited to: a high-resolution, digital, wand camera 102 (intra-oral camera) that can capture external tooth and gum features; a trans-illuminator 104 that can capture internal tooth features (such as fractures) with a high-intensity LED; a handheld X-ray probe 106 and a sonogram device 108 having one or more ultrasound sensors that provide contrasting light and dark segments of a tooth fracture. The intra-oral imaging devices shown in FIG. 1 may communicate with the device 100 wirelessly or wiredly, such that images taken through one or more of the intra-oral devices may be provided through the device 100 to a doctor device 110. As a non-limiting example, the system may be configured to allow a plurality of peripheral, intra-oral devices (micro handheld camera, trans-illuminator, x-ray and sonogram) to operate on a single mobile device using a software application that can recognize all devices within its vicinity by incorporating BlueTooth™, wireless technology. More than one intra-oral imaging device may be operated at the same time. The doctor device 110 may form at least part of a remote computing system associated (e.g., owned, operated, used by) with a dental professional/entity.

The imaging devices and/or the device 100 may be configured to record video, through the imaging devices such that a webcam chat may be established with the doctor device 110. For example, real-time video taken through via the trans-illuminator may be fed to the doctor device 110 in real time. It is to be understood that a stock light or camera on the device 100 may be used in capturing image data. For example, a stock flashlight on a smart phone may be used to capture trans-illumination images by using the stock flashlight to trans-illuminate teeth. Further, in some examples one or more of devices 102, 104, 106, and 108—and/or other devices used to capture dental image data such as a stock flashlight and camera—may be integrated in device 100.

The illustration of FIG. 1 further shows device 100 being connected to a cloud based network 112 and to a dentist's mobile device 110 through the cloud based network 112 (or internet). The device 100 also may be compatible with a variety of cloud server networks (shown as 112) as well as a multitude of dental-oriented software packages such as, but not limited to, Dexis, Eaglesoft, XDR, Apteryx, MacPractice, TigerView etc. The system may be configured to transmit secure communications using 256-bit encryption in compliance with all Health Insurance Portability and Accountability Act regulations.

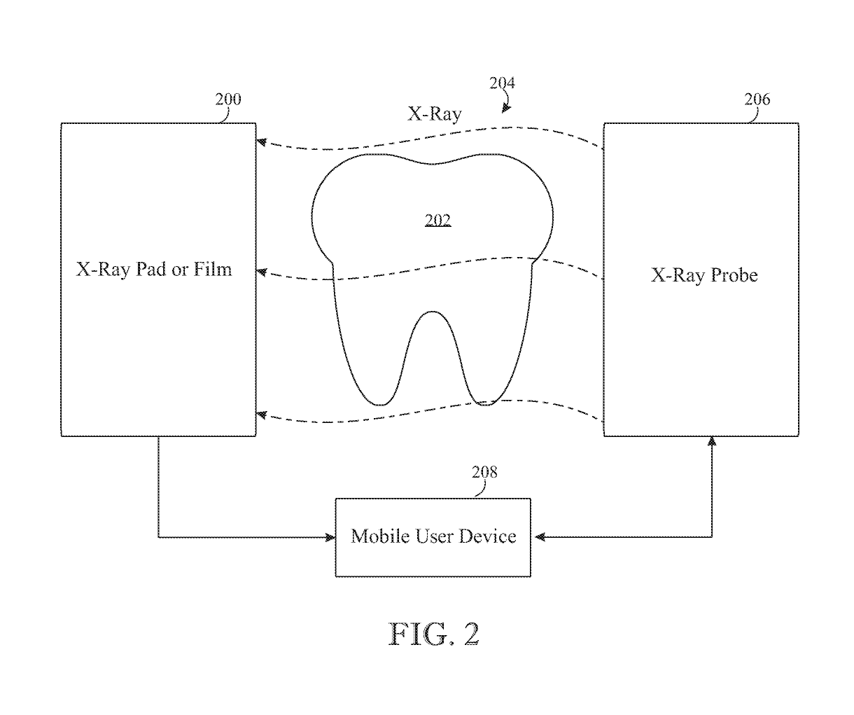

The illustration of FIG. 2 shows an embodiment of the system having a handheld X-Ray probe 206 transmitting X-Rays 204 though a tooth 202 and onto X-Ray pad 200 and the resulting exposure image from the X-Ray pad 200 being transmitted to a mobile device 208. It is to be understood that the mobile device 208 may be directly connected to the X-Ray probe 206 instead of the pad 200. The X-Ray probe 206 may be an X-Ray emitter, and/or an X-Ray camera that receives exposure images from the pad 200.

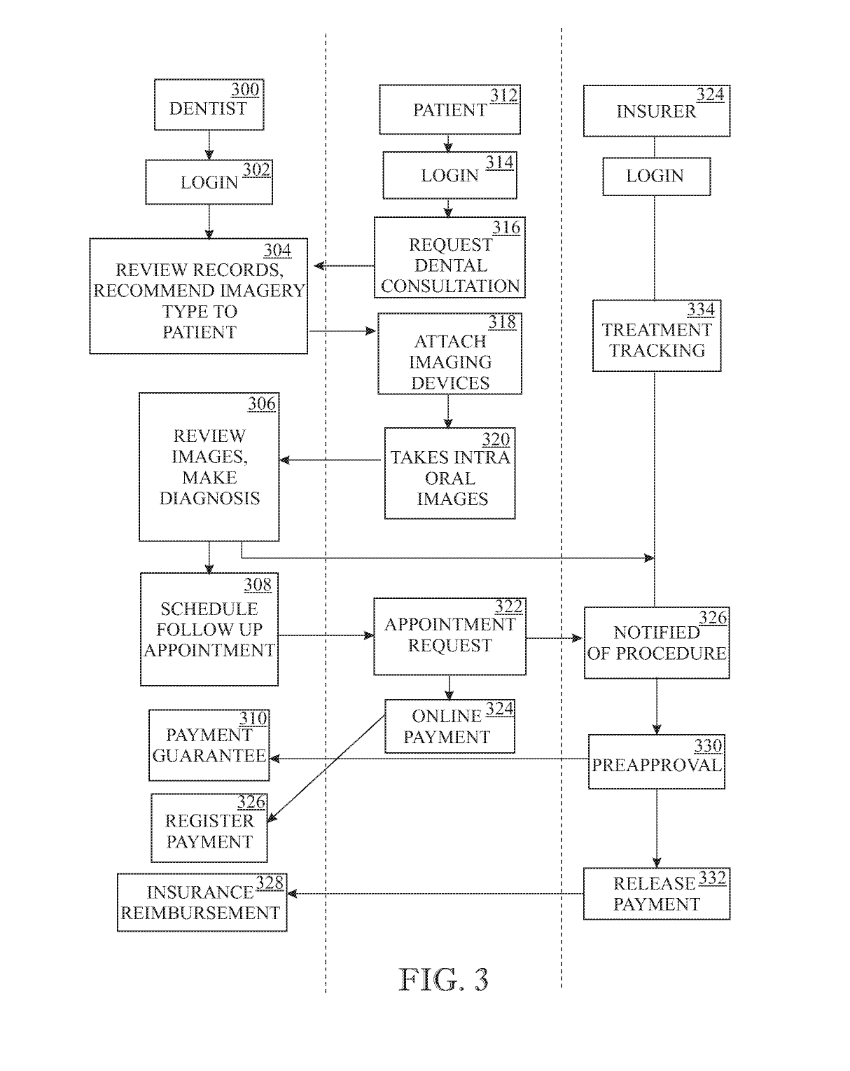

The illustration of FIG. 3 shows various steps being carried out via or using the system, according to user types. For example, for a patient user 312 the steps include logging in at 312, requesting a dental consult at 316, attaching peripheral imaging devices at 318, capturing images at 320 from the imaging devices, approving appointments on a calendar at 322, and making online dentist payments at 324. According to a dentist 300 user type, the steps include recommending imagery formats at 304 to patients remotely, performing online diagnoses at 306, scheduling follow-up appointments online at 308, receiving payment authorizations from insurers at 310, receiving online patient payments at 326, and requesting and receiving online insurance reimbursements at 328. According to an insurer 324 user type the steps may include, verifying treatment through tracking of patient dental activity at 334, receiving notifications of procedures from patients online at 326, performing pre-approvals at and sending preapprovals to dentists at 330, and releasing online payments to dentists at 332.

It is to be understood that requesting a dental consultation may include first filling out forms. For example, a patient may be required to enter demographic, medical history, and/or insurance information before a call to a dentist is initiated. The dentist may review the forms before recommending imaging modes at step 304.

Thus, the system may be configured to conduct remote, real-time consultations between dentist professionals and patients. For example, if a patient recovering from a procedure develops a painful infection, they can send images of their mouth using an application running on the mobile device 100 (connected to the host of intra-oral capture devices) directly to their dentist office or dentist device 110 and receive an immediate, remote consultation regarding the images. Generally, the device 100 may be configured to receive remote data from a remote computing system associated with a dental professional/entity, where the remote data indicates a diagnosis (or other medical opinion) made by the dental professional based on data collected by an intra-oral imaging device operatively coupled to the device 100.

As an example, the system may be configured to reduce costs incurred to a patient by minimizing unnecessary visits to the dentist. Patients who experience mouth discomfort (due to other common anomalies such as sinus congestion for example) can use the system to send intra-oral images to the dentist office and potentially rule out a costly, in-person examination.

As another example, the system may be configured to enhance patient self-efficacy. Patients who are equipped with the latest dental technology may feel part of the dental process, develop a greater sense of empowerment, and experience an increase in their ‘locus of control.’ Such enhancements have the potential to raise awareness of dental health and lead to an increase in overall societal public health over the long term.

As another example, the system may be configured to enhance patient monitoring. For example, dentists who perform complicated procedures can setup an online treatment monitoring and followup program using the disclosed system that includes sending a participating patient home with various, specialized portable imaging peripherals (micro handheld camera, trans-illuminator, x-ray and sonogram) and have patients capture intra-oral images with the borrowed devices and send the images via the system to the dentist professionals for review based on a predetermined schedule.

As another example, the system may be configured to enhance patient retention. Equipping a patient with dental monitoring tools and real-time remote consultations increases patient care, and as a result has the potential to make a positive impression on a patient and reinforce loyalty and retention.

As another example, the system may be configured to enhance patient record keeping by registering remote images captured by patients. For example, the system allows dentists to perform medical record keeping of captured images on a secured cloud network. The system also allows medical professionals to schedule appointments, send reminders, track patient concerns, file patient images on a cloud service and receive online payments. Remote, real time consultations also allow support teams better lead times to prepare an office for emergency response before a patient arrives.

As another example, the system may be configured to provide the insurance industry with enhanced claims support. Participating insurers can receive treatment updates and intra-oral images alongside dentist professionals in real time. Access to patient evidence such as intra-oral images can verify and justify proposed treatment plans and expedite the approval process and allow for more accurate risk-based forecasting.

As another example, the system may be configured to minimize potential, insurance fraud. For example, in the event a patient or dentist attempts acts of deception, the system can be used to request real-time verification of a dental condition and have a dentist (or in some cases even a patient) send imagery directly to an insurance investigator to rule out questionable services.

As another example, the system may be configured to reduce overall dental premiums for dentists and patients by minimizing unnecessary visits to the dentist. Reductions in face-to-face visits have the potential to curb group membership costs and lower aggregate premiums and may urge dentists to accept a wider range of insurance types leading to increased overall patient care in communities.

It is to be understood that the system may include a hardware device including any of the above described imaging devices, located at a dentist office, and configured for taking photos such that the photos are viewable through a patient mobile application, to allow people to take advantage of the system without having to acquire imaging hardware.

In conclusion disclosed is a remote dental consultation method and system compatible with a plurality of communication devices, tablets and intra-oral imaging devices. The system allows a multitude of teeth image types to be captured, transmitted and shared over a cloud-based network platform.

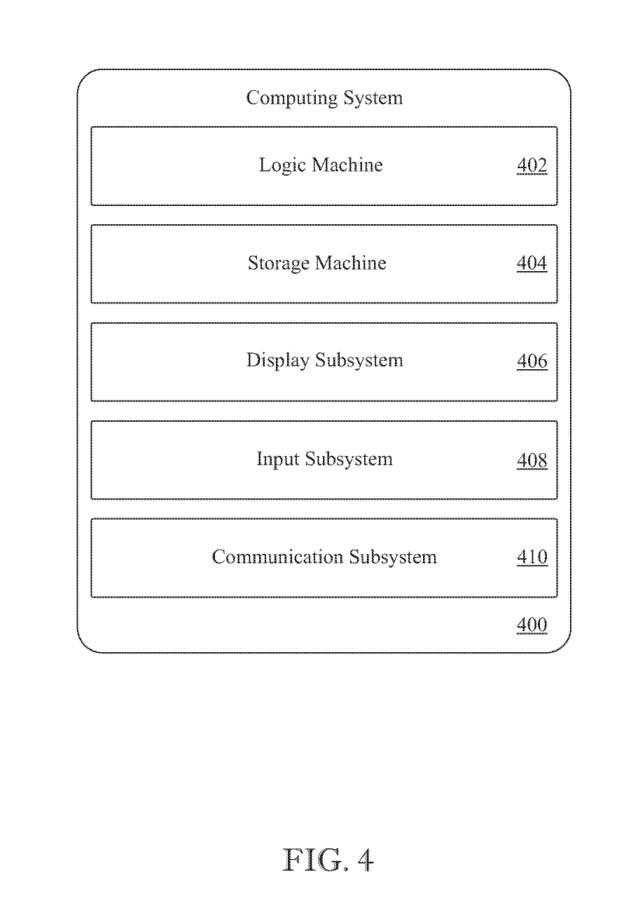

In some embodiments the methods, tasks, processes, and/or operations described above may be effected, executed, actualized, and/or carried out by a computing system including a tangible computer-readable storage medium, also described herein as a storage machine, that holds machine-readable instructions executable by a logic machine (i.e. a processor or programmable control device) to effect, execute, actualize, carry out, provide, implement, perform, and/or enact the above described methods, processes, operations, and/or tasks. For example, a suitable computing system may be computing system 400 shown in FIG. 4. When such methods, operations, and/or processes are implemented, the state of the storage machine 404 may be changed to hold different data. For example, the storage machine 404 may include memory devices such as various hard disk drives, CD, or DVD devices. The logic machine 402 may execute machine-readable instructions via one or more physical information and/or logic processing devices. For example, the logic machine 402 may be configured to execute instructions to perform tasks for a computer program. The logic machine 402 may include one or more processors to execute the machine-readable instructions. The computing system 400 may include a display subsystem 406 to display a graphical user interface (GUI) or any visual element of the methods or processes described above. For example, the display subsystem 406, storage machine 404, and logic machine 402 may be integrated such that the above method may be executed while visual elements of the disclosed system and/or method are displayed on a display screen for user consumption. The computing system 400 may include an input subsystem 408 that receives user input. The input subsystem 408 may be configured to connect to and receive input from devices such as a mouse, keyboard or gaming controller. For example, a user input may indicate a request that a certain task is to be executed by the computing system 400, such as requesting the computing system 400 to display any of the above described information, or requesting that the user input updates or modifies existing stored information for processing. A communication subsystem 410 may allow the methods described above to be executed or provided over a computer network. For example, the communication subsystem 410 may be configured to enable the computing system 400 to communicate with a plurality of personal computing devices. The communication subsystem 410 may include wired and/or wireless communication devices to facilitate networked communication. The described methods or processes may be executed, provided, or implemented for a user or one or more computing devices via a computer-program product such as via an application programming interface (API).

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A remote dental consultation system, the system comprising:
a personal electronic device; and
an intra-oral imaging device operatively connected to the personal electronic device;
wherein the personal electronic device is configured to communicate data collected by the intra-oral imaging device to a remote computing system.

2. The system of claim 1, wherein the remote computing system is associated with a dental professional.

3. The system of claim 1, wherein the intra-oral imaging device is configured to capture external tooth and gum features.

4. The system of claim 1, wherein the intra-oral imaging device is configured to capture internal tooth features.

5. The system of claim 1, wherein the intra-oral imaging device includes a digital intra-oral camera.

6. The system of claim 1, wherein the intra-oral imaging device includes a handheld X-Ray probe.

7. The system of claim 6, wherein the intra-oral imaging device further includes an X-Ray pad, and wherein the X-Ray probe is configured to transmit X-Rays through a tooth and onto the X-Ray pad to thereby generate an exposure image that is transmitted to the personal electronic device.

8. The system of claim 1, wherein the intra-oral imaging device includes a sonogram device having one or more ultrasound sensors.

9. The system of claim 1, wherein the intra-oral imaging device includes a trans-illuminator having a high-intensity LED.

10. The system of claim 1, wherein the personal electronic device is further configured to receive, from the remote computing system, remote data indicating a diagnosis made by a dental professional based on the data collected by the intra-oral imaging device.

11. The system of claim 1, wherein the data includes video captured by the intra-oral imaging device that is communicated to the remote computing system in real-time.

12. The system of claim 1, wherein the imaging device is at least partially integrated in the personal electronic device.

13. A personal electronic device, comprising:
a logic machine; and
a storage machine holding instructions executable by the logic machine to:
communicatively couple with an intra-oral imaging device;
receive image data from the intra-oral imaging device, the image data capturing one or more dental features of a user; and
transmit the image data to a remote computing system associated with a dental professional.

14. The personal electronic device of claim 12, wherein the one or more dental features include external tooth and gum features.

15. The personal electronic device of claim 12, wherein the one or more dental features include internal tooth features.

16. The personal electronic device of claim 12, wherein the intra-oral imaging device includes a digital intra-oral camera.

17. The personal electronic device of claim 12, wherein the intra-oral imaging device includes a handheld X-Ray probe.

18. The personal electronic device of claim 12, wherein the intra-oral imaging device includes a sonogram device having one or more ultrasound sensors.

19. The personal electronic device of claim 12, wherein the intra-oral imaging device includes a trans-illuminator having a high-intensity LED.

20. A remote dental consultation system, the system comprising:
a personal electronic device; and
an intra-oral imaging device operatively connected to the personal electronic device;
wherein the personal electronic device is configured to communicate image data capturing one or more dental features of a user collected by the intra-oral imaging device to a remote computing system associated with a dental professional, and to receive remote data from the remote computing system indicating a diagnosis made by the dental professional based on the data collected by the intra-oral imaging device.
